(19) United States
(12) Patent Application Publication
(10) Pub. No.: US 2019/0175293 A1
(43) Pub. Date: Jun. 13, 2019

(54) IMAGE GUIDANCE FOR A DECOUPLED KINEMATIC CONTROL OF A REMOTE-CENTER-OF-MOTION

(71) Applicant: KONINKLIJKE PHILIPS N.V., EINDHOVEN (NL)

(72) Inventors: Aleksandra POPOVIC, BOSTON, MA (US); David Paul NOONAN, NEW YORK, NY (US)

(21) Appl. No.: 16/309,840

(22) PCT Filed: Jun. 22, 2017
(86) PCT No.: PCT/EP2017/065381

§ 371 Date: Dec. 13, 2018

Related U.S. Application Data
(60) Provisional application No. 62/353,328, filed on Jun. 22, 2016.

(51) Int. Cl. A61B 34/30; A61B 90/50; A61B 34/00; B25J 9/16

(52) U.S. Cl. CPC A61B 34/30; A61B 90/50; A61B 2034/2059; B25J 9/1689; A61B 34/70

(57) ABSTRACT

A robotic system employing a robotic apparatus and a robot controller (20) for executing an interventional procedure. The robotic apparatus includes a robot manipulator (30) and an intervention robot (40) mounted to the robot manipulator (30) with a structural configuration of the intervention robot (40) defining a remote-center-of-motion. The robot controller (20) controls a manual actuation of a translational motion and/or a rotational motion of the robot manipulator (30) directed to a spatial positioning of the intervention robot (40) within a kinematic space of the robot manipulator (30) derived from a delineation of spatial positioning of the remote-center-of-motion within an image space. The robot controller (20) further controls a signal actuation of a pitch motion and/or a yaw motion of the intervention robot (40) directed to a spatial orienting of the end-effector within a kinematic space of the intervention robot (40) derived from a delineation of a spatial orienting of the remote-center-of-motion within the image space.

### FIELD OF THE INVENTION

The present disclosure generally relates to robots utilized during various interventional procedures (e.g., laparoscopic surgery, neurosurgery, spinal surgery, natural orifice transluminal surgery, pulmonary/bronchoscopy surgery, biopsy, ablation, and diagnostic interventions). The present disclosure specifically relates to an image guidance of a decoupled spatial positioning and spatial orienting control of an intervention robot.

### BACKGROUND OF THE INVENTION

Minimally invasive surgery is performed using elongated instruments inserted into the patient's body through small ports. More particularly, the small ports that are placed on the patient's body are the only incision points through which the instruments may pass through to access the inside of the patient. As such, the instruments may be operated to rotate around these fulcrum points, but the instruments should not be operated in a manner that imposes translational forces on the ports to prevent any potential injury and harm to the patient. This is especially important for robotic guided surgery.

To the end, some known robots implement what is known as a remote-center-of-motion (RCM) at the fulcrum point whereby a robot enforces an operating principle that only rotation of an instrument can be performed at a port and all translational forces of the instrument at that port are eliminated. This can be achieved by implementing a mechanical design which has the RCM at a specific location in space, and then aligning that point in space with the port. Alternatively, the RCM can be implemented virtually within the software of a robotic system, provided sufficient degrees of freedom exist to ensure the constraints of the RCM can be met.

Constraint robots, such as RCM robots, are challenging to control. Such robots usually implement at least five (5) joints of which (3) joints are used to position the RCM and at least two (2) joint are used to orient the RCM. Due to kinematic constraints, mapping between the joints and space degrees of freedom is not intuitive. Furthermore, the safety of these such robots can be compromised if the user accidentally moves the RCM after the instrument is inserted into the patient body. The computationally constraint systems for such robots are even more difficult to operate as those constraints are less intuitive.

### SUMMARY OF THE INVENTION

The present disclosure provides a control of a robotic apparatus employing a robot manipulator and an intervention robot whereby the control utilizes image guidance to independently control the robot manipulator and the intervention robot for a spatial positioning and a spatial orienting, respectively, of the intervention robot. For example, during a surgical intervention, the robotic apparatus is controlled by image guidance for a manual actuation of the robot manipulator to independently spatially position the intervention robot to coincide with an insertion point into a body as supported by an operating table serving as a reference plane, and for a signal actuation of the intervention to independently spatially orient an end-effector of the intervention robot to orient an intervention instrument supported by the intervention robot in an intuitive view of the operating table again serving as the reference plane.

One form of the inventions of the present disclosure is a robotic system employing a robotic apparatus and a robot controller for executing an interventional procedure.

The robotic apparatus includes an intervention robot mounted unto a robot manipulator. A structural configuration of the intervention robot defines a remote-center-of-motion (RCM).

For a spatial positioning of the intervention robot, the robot controller controls a manual actuation of a translational motion and/or a rotational motion of the robot manipulator directed to a spatial positioning of the intervention robot within a kinematic space of the robot manipulator derived from a delineation of a spatial positioning of the remote-center-of-motion within an image space.

For a spatial orienting of the an end-effector of the intervention robot, the robot controller controls a signal actuation of a pitch motion and/or a yaw motion of the intervention robot directed to a spatial orienting of the end-effector of the intervention robot within a kinematic space of the intervention robot derived from a delineation of a spatial orienting of the remote-center-of-motion within the image space.

A second form of the inventions of the present disclosure is a control network including the robot controller and further including an image controller controlling a communication to the robot controller of the delineations of the spatial positioning and the spatial orienting of the remote-center-of-motion within the image space

A third form of the inventions of the present disclosure is a method for controlling the robot manipulator and the intervention robot of the robot apparatus.

For a spatial positioning of the intervention robot, the method involves the robot controller controlling a manual actuation of a translational motion and/or a rotational motion of the robot manipulator directed to a spatial positioning of the intervention robot within a kinematic space of the robot manipulator derived from a delineation of a spatial positioning of the remote-center-of-motion within an image space.

For a spatial orienting of the end-effector of the intervention robot, the method further involves the robot controller controlling a signal actuation of a pitch motion and/or a yaw motion of the intervention robot directed to a spatial orienting of the end-effector of the intervention robot within a kinematic space of the intervention robot derived from a delineation of a spatial orienting of the remote-center-of-motion within the image space.

For purposes of the present disclosure,

(1) the term “robot manipulator” broadly encompasses any mechanical device having a structural configuration, as understood in the art of the present disclosure and as exemplary described herein, one or more articulated joints (e.g., prismatic joints and/or revolute joints) capable of a manual actuation of a translational motion and/or a rotational motion of segments and/or links in one or more degrees of freedom;

(2) the term “manual actuation” broadly encompasses, as understood in the art of the present disclosure and as exemplary described herein, an operator of the robot manipulator utilizing hands, mechanical device(s), etc. to actuate the translational motion and/or the rotational motion of the segments and/or links in one or more degrees of freedom;

(3) the phrase “kinematic space of the robot manipulator” broadly encompasses, as understood in the art of the present disclosure and as exemplary described herein, a spatial area traversable by the intervention robot over a range of translational motion and/or a range of rotational motion of the robot manipulator;

(4) the term “intervention robot” broadly encompasses any robot having a structural configuration, as understood in the art of the present disclosure and as exemplary described herein, including two or more revolute joints and an end-effector whereby an intersection of axes of the revolute joints and the end-effector defines a remote-center-of-motion at a fulcrum point in space whereby an instrument held by the end-effector may be pitched, yawed and/or rolled at the remote-center-of-motion;

(5) the term “signal actuation” broadly encompasses, as understood in the art of the present disclosure and as exemplary described herein, an application of a signal to the revolute joints of the intervention robot to thereby drive an actuation of the pitch motion and/or the yaw motion of the intervention robot;

(6) the phrase “kinematic space of the intervention robot” broadly encompasses, as understood in the art of the present disclosure and as exemplary described herein, a spatial area enclosing a range of pitch motion and/or a range of yaw motion of the intervention robot;

(7) the phrase “a delineation of a spatial positioning of the remote-center-of-motion within the image space” broadly encompasses, as understood in the art of the present disclosure and as exemplary described herein, a user delineation of a position of the remote-center-of-motion within a diagnostic image whereby the user delineation corresponds to a desired insertion point into a patient illustrated in the diagnostic image;

(8) the phrase “a delineation of a spatial orienting of the remote-center-of-motion within the image space” broadly encompasses, as understood in the art of the present disclosure and as exemplary described herein, a user delineation of an orientation of the remote-center-of-motion within a diagnostic image whereby the user delineation corresponds to a desired axial orientation of an end-effector of the intervention robot relative to a desired insertion point into a patient illustrated in the diagnostic image or whereby the user delineation corresponds to a desired axial orientation of an intervention tool supported by the end-effector of the intervention robot relative to the desired insertion point into the patient illustrated in the diagnostic image;

(9) the term “image space” broadly encompasses, as understood in the art of the present disclosure and as exemplary described herein, a spatial area imaged by a imaging modality;

(10) the term “controller” broadly encompasses all structural configurations, as understood in the art of the present disclosure and as exemplary described herein, of an application specific main board or an application specific integrated circuit for controlling an application of various inventive principles of the present disclosure as subsequently described herein. The structural configuration of the controller may include, but is not limited to, processor(s), computer-usable/computer readable storage medium(s), an operating system, application module(s), peripheral device controller(s), slot(s) and port(s). A controller may be housed or linked to a workstation. Examples of a “workstation” include, but are not limited to, an assembly of one or more computing devices, a display/monitor, and one or more input devices (e.g., a keyboard, joysticks and mouse) in the form of a standalone computing system, a client computer, a desktop or a tablet.

(11) the descriptive labels for term “controller” herein facilitates a distinction between controllers as described and claimed herein without specifying or implying any additional limitation to the term “controller”;

(12) the term “module” broadly encompasses a module incorporated within or accessible by a controller consisting of an electronic circuit and/or an executable program (e.g., executable software stored on non-transitory computer readable medium(s) and/firmware) for executing a specific application;

(13) the descriptive labels for term “module” herein facilitates a distinction between modules as described and claimed herein without specifying or implying any additional limitation to the term “module”;

(14) the terms “signal” and “data” broadly encompasses all forms of a detectable physical quantity or impulse (e.g., voltage, current, or magnetic field strength) as understood in the art of the present disclosure and as exemplary described herein for transmitting information in support of applying various inventive principles of the present disclosure as subsequently described herein;

(15) the descriptive labels for term “signal” herein facilitates a distinction between signals as described and claimed herein without specifying or implying any additional limitation to the term “signal”; and

(16) the descriptive labels for term “data” herein facilitates a distinction between data as described and claimed herein without specifying or implying any additional limitation to the term “data”.

The foregoing forms and other forms of the inventions of the present disclosure as well as various features and advantages of the present disclosure will become further apparent from the following detailed description of various embodiments of the present disclosure read in conjunction with the accompanying drawings. The detailed description and drawings are merely illustrative of the present disclosure rather than limiting, the scope of the present disclosure being defined by the appended claims and equivalents thereof.

### BRIEF DESCRIPTION OF THE DRAWINGS

FIG. 1 illustrates a block diagram of an exemplary embodiment of a robotic system in accordance with the inventive principles of the present disclosure.

FIG. 2 illustrates block diagrams of a first exemplary embodiment of an image controller and a robot controller in accordance with the inventive principles of the present disclosure.

FIG. 3 illustrates block diagrams of a second exemplary embodiment of an image controller and a robot controller in accordance with the inventive principles of the present disclosure.

FIGS. 4A and 4B illustrates a side view and a top view, respectively, of a schematic diagram of an exemplary embodiment of a robot manipulator in accordance with the inventive principles of the present disclosure.

FIG. 5A-5D illustrates exemplary embodiments of various position indicators in accordance with the inventive principles of the present disclosure.

FIG. 6 illustrates an exemplary embodiment of an intervention robot as known in the art.

FIG. 7 illustrates an exemplary interventional procedure in accordance with the inventive principles of the present disclosure.

FIG. 8 illustrates a flowchart representative of an exemplary embodiment of a robot apparatus control method in accordance with the inventive principles of the present disclosure.

FIG. 9A illustrates a flowchart representative of an exemplary embodiment of a robot manipulator control method in accordance with the inventive principles of the present disclosure.

FIG. 9B illustrates a flowchart representative of an exemplary embodiment of an intervention robot control method in accordance with the inventive principles of the present disclosure.

### DETAILED DESCRIPTION OF THE PREFERRED EMBODIMENTS

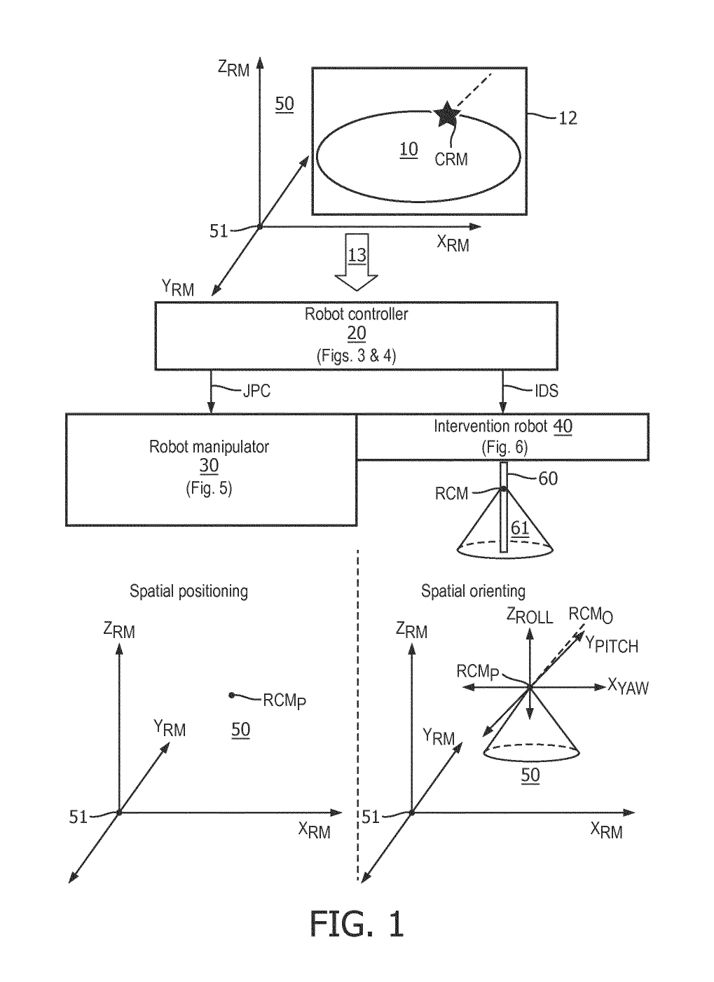

To facilitate an understanding of the present disclosure, the following description of FIG. 1 teaches basic inventive principles of a robotic apparatus employing a robot manipulator and an intervention robot, and a robotic control method implementing an image guidance to independently control a manual actuation of the robot manipulator for a desired spatial positioning of the intervention robot as user delineated within an image space, and to further control a signal actuation of the intervention robot for a desired spatial orienting of an end-effector of the intervention robot, particularly a desired spatial orienting of an end-effector tool of the intervention robot or of an intervention tool supported by the end-effector, as user delineated within an image space. From this description, those having ordinary skill in the art will appreciate how to apply the inventive principles of the present disclosure to numerous and various types of interventional procedures.

Referring to FIG. 1, a robotic system of the present disclosure employs a robot controller 20, and a robotic apparatus including an intervention robot 40 removably or permanently mounted to a robot manipulator 30.

Robot controller 20 receives an input 13 illustrative/informative of a user delineation of a position of the remote-center-of-motion RCM within a diagnostic image 12 whereby the user delineation corresponds to a desired insertion point into a patient 10 illustrated in diagnostic image 12. Input 13 is further illustrative/informative of a user delineation of an orientation of the remote-center-of-motion RCM within diagnostic image 12 whereby the user delineation corresponds to a desired axial orientation of an end-effector of intervention robot 40 relative to the desired insertion point into patient P illustrated in diagnostic image 12, or whereby the user delineation corresponds to a desired axial orientation of an intervention tool supported by the end-effector of intervention robot 40 relative to the desired insertion point into patient P illustrated in diagnostic image 12.

In practice, any type of imaging modality as known in the art suitable for an interventional procedure may be utilized to generate diagnostic image 12 (e.g., a computed-tomography (CT) modality, a magnetic resonance imaging (MRI) modality, an X-ray modality, an ultrasound (US) modality, etc.). Also in practice, input 13 may be have any form suitable for communicating the spatial positioning and spatial orienting of intervention robot 40 as delineated within the image space of diagnostic image 12 including, but not limited to, image data corresponding to diagnostic image 12 or coordinate data informative of the spatial positioning and spatial orienting of the remote-center-of-motion RCM within the image space of diagnostic image 12 as registered to a kinematic space 50 of robot manipulator 30.

Robot controller 20 processes input 13 to independently control a manual actuation of robot manipulator 30 via joint position command(s) JPC as will be further described herein, or to independently control a signal actuation of intervention robot 40 via an interventional drive signal IDS as will be further described herein.

In practice, robot controller 20 may be housed within or linked to a workstation wired to or wirelessly connected to the imaging modality, or may be housed within a workstation of the imaging modality.

Robot manipulator 30 includes one (1) or more articulated joints (not shown) (e.g., prismatic joint(s), and/or revolute joint(s)) providing one (1) or more degrees of freedom for translational motion and/or rotational motion of segments/links and end-effector (not shown) of robot manipulator 30 as manually actuated by an operator of robot manipulator 30 in accordance with joint position command(s) JPC. A range of translational motion and/or a range of rotational motion of the segment/links and end-effector define a kinematic space of robot manipulator 30 as will be further described herein.

In one embodiment of robot manipulator 30, one or more articulated joints extend between a base segment/link, and an end-effector for mounting intervention robot 40 upon robot manipulator 30. For this embodiment, any translational motion and/or rotational motion of the segments/links and end-effector of robot manipulator 30 is based on the base segment/link serving as a point of origin of the kinematic space of robot manipulator 30.

Intervention robot 40 includes one (1) or more arms and/or arcs (not shown) supporting two (2) or more actuators (not shown) in a structural configuration having rotational axes of the actuators intersecting in at a fulcrum point within a kinematic space of intervention robot 40 defining the remote-center-of-motion RCM as will be further described herein. Intervention robot 40 further includes an end-effector (not shown) for holding an intervention instrument whereby the remote-center-of-motion RCM is positioned along an axis of the end-effector and/or an axis of the intervention instrument to establish a workspace defined by motion of the intervention instrument.

For example, the end-effector of intervention robot 40 holds an intervention instrument 60 whereby the remote-center-of-motion RCM is positioned along a longitudinal axis of intervention instrument 60 having a workspace 61. In practice, a specified embodiment of intervention instrument is dependent upon the particular interventional procedure being executed by the robotic system. Examples of an intervention instrument include, but are not limited to, surgical instruments and viewing/imaging instruments (e.g., an endo scope).

Still referring to FIG. 1, a spatial positioning operation generally involves robot controller 20 controlling a manual actuation of spatial positioning of intervention robot 40 at a coordinate point within the kinematic space of robot manipulator 30 that corresponds to a delineated spatial position of the remote-center-of-motion RCM of intervention robot 40 at a coordinate point within the image space of diagnostic image 12. Intervention robot 40 is removably or permanently mounted in any suitable manner to robot manipulator 30 to move in unison with any manual actuation by an operator of robot manipulator 30. Upon reaching the directed coordinate point, a spatially orienting operation generally involves robot controller 20 controlling a signal actuation of intervention robot 40 as needed to spatially orient an end-effector of intervention robot 40 at an orientation within a kinematic space of intervention robot 40 that is registered to the spatial orientation of the remote-center-of-motion RCM of intervention robot 40 about the coordinate point within the image space of diagnostic image 12.

For example, an exemplary operation of the robotic system is shown in FIG. 1. Note intervention robot 40 mounted to robot manipulator 30 is not shown for visual clarity in the description of an exemplary spatial positioning operation and an exemplary spatial orienting operation of the robotic system. Nonetheless, those skilled in the art will appreciate the remote-center-of-motion RCM is symbolic of robot manipulator 30 and intervention robot 40 for purposes of the exemplary operation.

In the example, a spatial positioning by the operator of intervention robot 40 being positioned at coordinate point within a kinematic space 50 of robot manipulator 30 symbolized by a coordinate system X_{MR}-Y_{MR}-Z_{MR} having a point of origin 51 involves:
(1) robot controller 20 processing input 13 to identify a coordinate point RCM_{P} within kinematic space 50 of robot manipulator 30 that corresponds to a delineated spatial position of the remote-center-of-motion RCM of intervention robot 40 at a coordinate point within the image space of diagnostic image 12, and
(2) robot controller 20 communicating the joint position command(s) JPC informative of a position setting of one or more articulated joints of robot manipulator 30 for a manual actuation of any translational motion and/or rotational motion of the articulated joint(s) necessary to thereby spatially position the remote-center-of-motion RCM of intervention robot 40 at coordinate point RCM_{P}.

In practice, a joint position command JPC may be in any suitable form for communicating the position setting of one or more articulated joints of robot manipulator 30 including, but not limited to, a textual display of a joint position setting, an audible broadcast of a joint position setting, and a graphical image of the joint position setting as will be further described herein.

Also by example as shown in FIG. 1, a spatial orienting of an end-effector of intervention robot 40 at an orientation about the coordinate point RCM_{P} within a kinematic system of intervention robot 40 symbolized by coordinate system X_{YAW}-axis, a Y_{PITCH}-axis and a Z_{ROLL}-axis involves:
(1) robot controller 20 processing input 13 to identify an orientation of an end-effector of intervention robot 40 or an intervention instrument supported by the end-effector, about the coordinate point RCM_{P} that corresponds to a delineated spatial orientation of the remote-center-of-motion RCM of intervention robot 40 at a coordinate point within the image space of diagnostic image 12, and
(2) robot controller 20 transmitting an interventional drive signal IDS to intervention robot 40 for a signal actuation of a pitch motion and/or yaw motion of the arms/arcs of intervention robot 40 to spatially orient the end-effector of intervention robot 40 at orientation RCM_{O} about coordinate point RCM_{P} within the kinematic space of intervention robot 40.

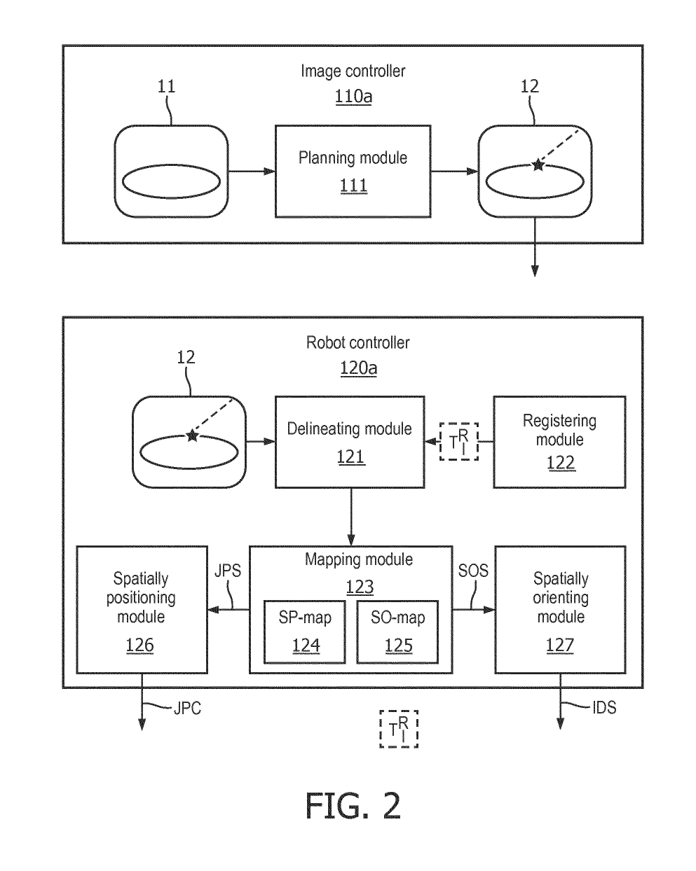

To further facilitate an understanding of the present disclosure, the following description of FIGS. 2-6 describes exemplary embodiments of an image controller (not shown in FIG. 1), robot controller 20, robot manipulator 30 and intervention robot 40 for practicing the basic inventive principles of FIG. 1. From this description, those having ordinary skill in the art will appreciate how to apply the inventive principles of the present invention to numerous and various embodiments of user input device 10, robot controller 20, robot manipulator 30 and intervention robot 40.

Referring to FIG. 2, an embodiment 120a of robot controller 20 (FIG. 1) employs a delineating module 121, a registering module 122, a mapping module 123, a spatial positioning module 125 and a spatial orienting module 126 for processing image data corresponding to diagnostic image 12 or coordinate data informative of the spatial positioning and spatial orienting of the remote-center-of-motion RCM of intervention robot 40 within the image space of diagnostic image 12 as registered to a kinematic space 50 of robot manipulator 30.

Specifically, an image controller 110a employs a planning module 111 implementing known planning techniques of the art for planning in insertion point and insertion angle of an intervention instrument within a diagnostic image 11 of a patient 10 generated by an imaging modality (e.g., CT, cone-beam CT, MRI, X-ray, US, etc.) to thereby generate diagnostic image 12.

Image controller 110a communicates a live version or a stored version of diagnostic image 12 to robot controller 120a whereby delineating module 121 generates registered robot data RRD informative of a spatial positioning and a spatial orienting of intervention robot 40 within the respective kinematic spaces of robot manipulator 30 and intervention robot 40. More particularly, registering module 122 generates a transformation matrix T based on a registration by any known technique of robot manipulator 30 and intervention robot 40 as a single robot apparatus to the imaging modality of diagnostic image 11, and delineating module 121 generates registered robot data RRD by applying transformation matrix T to diagnostic image 12.

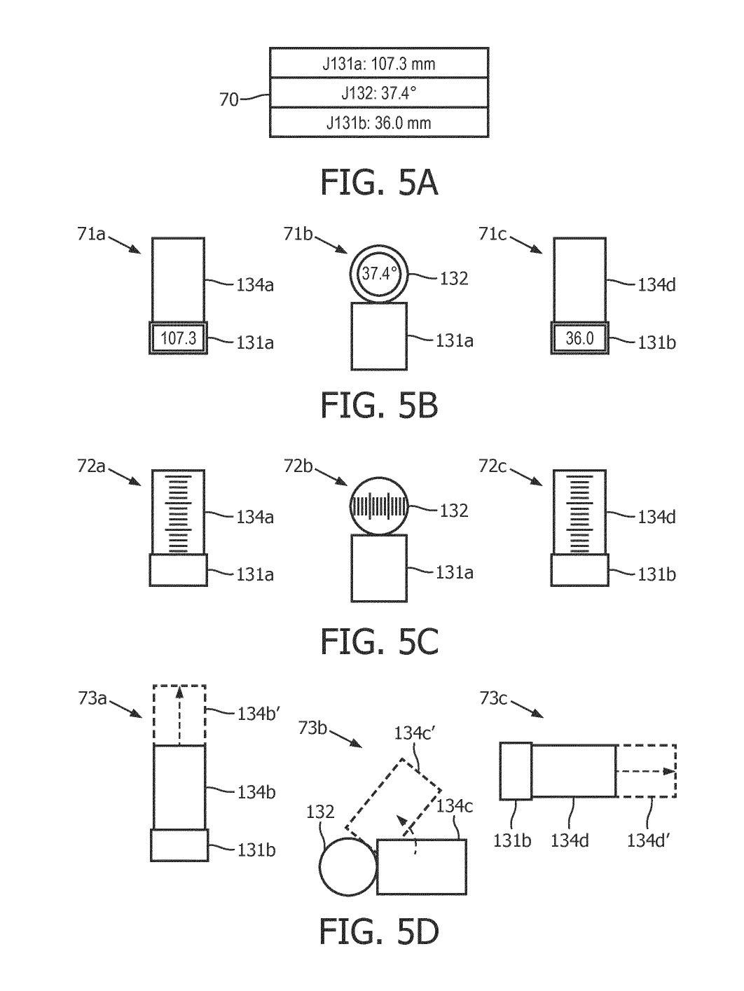

Mapping module 123 includes a spatial positioning map 124 for processing the spatial position information of registered robot data RRD to thereby generate joint position settings JPS informative of a position of each articulated joint of robot manipulator 50 for spatially positioning intervention robot 40 within the kinematic space of robot manipulator 30. In response thereto, spatial positioning module 126 generates joint position command(s) JPC (e.g., a textual display, an audible broadcast and/or graphical image) for any necessary manual actuation of a translational motion and/or a rotational motion of a robot manipulator 130 (FIG. 5) as will be further described herein.

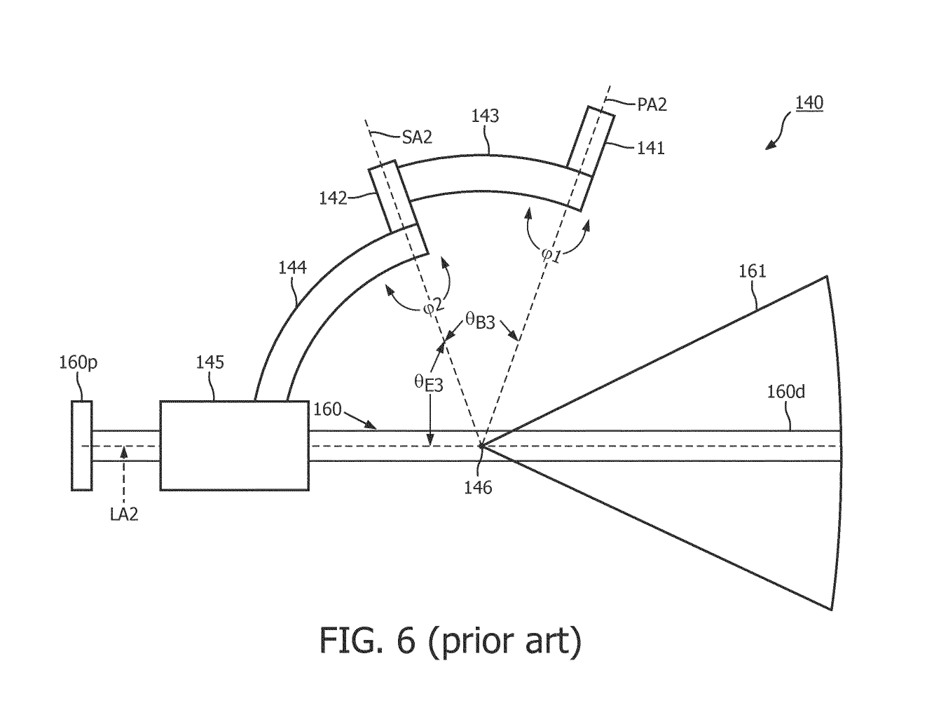

Mapping module 123 further includes a spatial orienting map 125 for processing the spatial orientation information of registered robot data RRD to thereby generate a spatial orienting signal SOS as any necessary angular vector transformation of the spatial orientation information as will be further described herein. In response thereto, spatial orientating module 127 generate an interventional drive signal IDS for driving a pitch motion and/or a yaw motion of an intervention robot 140 (FIG. 6) as will be further described herein.

In practice, image controller 110a and robot controller 120a may be separate controllers housed or linked to the same workstation or different workstations, or may be integrated into a single master controller housed or linked to the same workstation. For example, image controller 110a may be housed within a workstation of the imaging modality, and robot controller 120a may be housed within a workstation of the robotic apparatus.

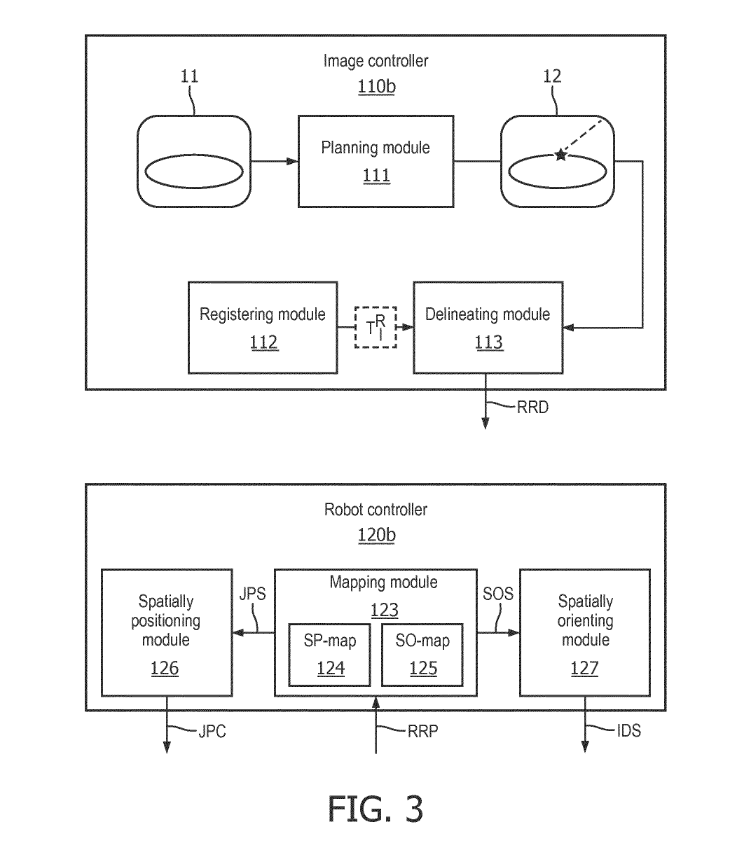

Referring to FIG. 3, an embodiment 120b of robot controller 20 (FIG. 1) employs mapping module 123, spatial positioning module 125 and spatial orienting module 126 for processing registered robot data RRD as previously described for FIG. 2. For robot controller 120b, an image controller 110 employs planning module 111, a registering module 112 and a delineating module 113 for generating registered robot data RD as previously described for FIG. 2.

In practice, image controller 110b and robot controller 120b may be separate controllers housed or linked to the same workstation or different workstations, or may be integrated into a single master controller housed or linked to the same workstation. For example, image controller 110b may be housed within a workstation of the imaging modality, and robot controller 120b may be housed within a workstation of the robotic apparatus.

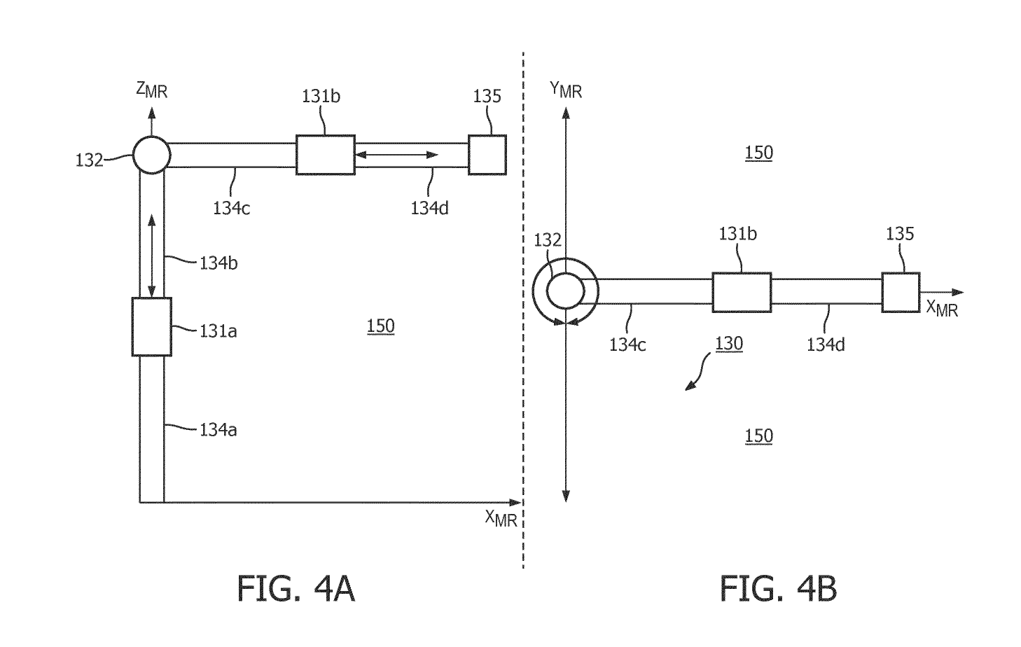

Referring to FIGS. 4A and 4B, an embodiment 130 of robot manipulator 30 (FIG. 1) employs a prismatic joint 131a connecting rigid links 134a and 134b, a revolute joint 132 connecting rigid links 134b and 134c, a prismatic joint 131b connecting rigid links 134c and 134d, and an end-effector 135 for removably or permanently mounting of intervention robot 140 (FIG. 5) thereon.

In operation, link 134a serves as a base link for a point of origin of a kinematic space 150 of robot manipulator 130. When manually actuated by an operator of robot manipulator 130, prismatic joint 131a translationally moves links 134b, 134c and 134d and end-effector 135 in unison along the Z-axis of kinematic space 150 of robot manipulator 130 as best shown in FIG. 4A.

When manually actuated by an operator of robot manipulator 130, revolute joint 132 rotationally moves 134c and 134d and end-effector 135 in unison about the Z-axis of kinematic space 150 of robot manipulator 130 as best shown in FIG. 4B.

When manually actuated by an operator of robot manipulator 130, prismatic joint 131b translationally moves link 134d and end-effector 135 in unison along the X-axis and/or the Y-axis of kinematic space 150 robot manipulator 130 as shown in FIGS. 4A and 4B.

Referring to FIG. 5A, spatially positioning module 126 may control a textual display 70 of a target joint position of prismatic joints 131a and 131b and revolute joint 132.

From textual display 70, prismatic joint 131a may employ a linear encoder for a textual indication 71a of a current joint position of prismatic joint 131a as shown in FIG. 5B whereby an operator of robot manipulator 130 may ascertain any necessary translational motion of prismatic joint 131a to reach the target joint position of prismatic joint 131a.

Similarly, revolute joint 132 may employ a rotary encoder for a textual indication 71b of a current joint position of revolute joint 132 as shown in FIG. 5B whereby an operator of robot manipulator 130 may ascertain any necessary rotational motion revolute joint 132 to reach the target joint position of revolute joint 132.

Similarly, prismatic joint 131b may employ a linear encoder for a textual display 71c of a current joint position of prismatic joint 131b as shown in FIG. 5B whereby an operator of robot manipulator 130 may ascertain any necessary translational motion of prismatic joint 131b to reach the target joint position of prismatic joint 131b.

Alternatively from textual display 70, prismatic joint 131a may employ a measurement scale for a textual indication 72a of a current joint position of prismatic joint 131a as shown in FIG. 5C whereby an operator of robot manipulator 130 may ascertain any necessary translational motion of prismatic joint 131a to reach the target joint position of prismatic joint 131a.

Similarly, revolute joint 132 may employ a measurement scale for a textual indication 72b of a current joint position of revolute joint 132 as shown in FIG. 5C whereby an operator of robot manipulator 130 may ascertain any necessary rotational motion revolute joint 132 to reach the target joint position of revolute joint 132.

Similarly, prismatic joint 131b may employ measurement markers for a textual display 72c of a current joint position of prismatic joint 131b as shown in FIG. 5C whereby an operator of robot manipulator 130 may ascertain any necessary translational motion of prismatic joint 131b to reach the target joint position of prismatic joint 131b.

Referring to FIG. 5D, alternate to textual display 70, a graphical image 73a may be displayed as a visual indication of a relative distance between a current joint position of prismatic joint 131a and a target joint position 134b′, a graphical image 73b may be displayed as a visual indication of a relative distance between a current joint position of revolute joint 132 and a target joint position 134c′, and a graphical image 73c may be displayed as a visual indication of a relative distance between a current joint position of prismatic joint 131b and a target joint position 134d′. Graphical images are updated as joints are moved to reach the target positions.

Referring to FIG. 6, an embodiment 140 of intervention robot 40 (FIG. 1) employs an revolute joint 141 having a primary axis PA2, an revolute joint 142 having a secondary axis SA2, a support arc 143, and an instrument arc 144 including an end-effector 145 for holding an endoscope 160 having a longitudinal axis LA2. Support arc 143 is concentrically connected to revolute joint 141 and revolute joint 142, and instrument arc 144 is concentrically connected to revolute joint 142. Of importance,
(1) rotational axes PA2, RAD and LA2 intersect at a remote-center-of-motion 146,
(2) a base arch length of θ_{B} of support arc 143 extends between rotation axes PA2 and SA2,
(3) an extension arc length θ_{E3} of instrument arc 144a extends between rotation axes PA2 and LA2,
(4) a range of pitch motion and a range of yaw motion of end-effector 145 about remote-center-of motion 146 defines a kinematic space of intervention robot 140,
(5) a workspace 161 relative to remote-center-of-motion 146 has surface and base dimensions derived from base arch length of θ_{B3} of support arc 143 and extension arc length θ_{E3} of instrument arc 144a,
(6) revolute joint 141 may be driven by the robot controller as known in the art to co-rotate arcs 143 and 144a about primary axis PA2 for a desired (pi degrees to control a broad movement of a distal tip 160d of endoscope 160 within workspace 161,
(7) revolute joint 142 may be driven by the robot controller as known in the art to rotate instrument arc 144 about secondary axis SA2 for a desired φ₂ degrees to control a targeted movement of distal tip 160d of endoscope 160 within workspace 161, and
(8) end effector 161 has a capability, manual or controlled by the robot controller, of rotating endoscope 160 about its longitudinal axis LA2.

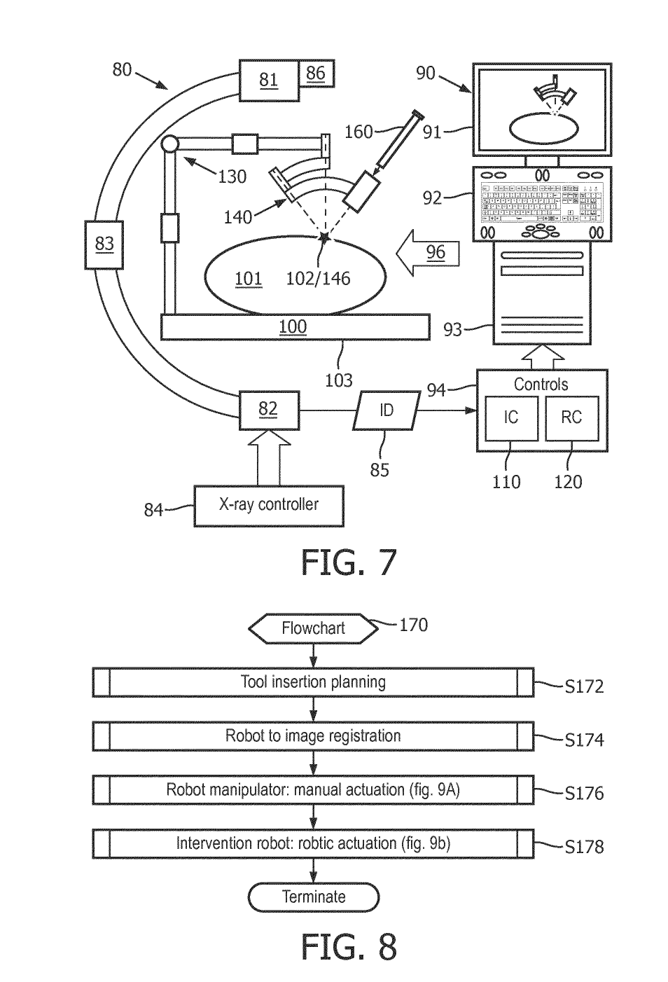

To facilitate a further understanding of the inventive principles of the present disclosure, image controller 110 (FIGS. 2 and 3), robot controller 120 (FIGS. 2 and 3), robot manipulator 130 (FIG. 4) and intervention robot 140 (FIG. 6) within a surgical environment will now be described herein in connection with FIGS. 7-9. From the description, those having ordinary skill in the art will appreciate how to operate numerous and various embodiments of an image controller, a robot controller, a robot manipulator and an intervention robot within any type of operational environment in accordance with the inventive principles of the present disclosure.

Referring to FIG. 7, the surgical environment includes an image controller 110, a robot controller 120, robot manipulator 130, and intervention robot 140 as previously described herein, and additionally includes an interventional X-ray imager 100, an intervention tool 160 (e.g., a surgical instrument) and a workstation 90 employing a monitor 91, a keyboard 92 and a computer 93.

As known in the art, interventional X-ray imager 80 generally includes an X-ray source 81, an image detector 82 and a collar 83 for rotating interventional X-ray imager 80. In operation as known in the art, an X-ray controller 84 controls a generation by interventional X-ray imager 80 of imaging data 85 illustrative of a cone-beam CT image of the anatomical object of a patient 101.

In practice, X-ray controller 84 may be installed within an X-ray imaging workstation (not shown), or alternatively installed within workstation 90.

Also in practice, interventional X-ray imager 80 may further employ a camera 86 rigidly attached to the C-arm with a known transformation between a camera coordinate system and the C-arm. The surgical procedure involves a spatial positioning and a spatial orienting of the remote-center-of-motion 146 of intervention robot 140 to coincide with an insertion port 102 of patient 101 resting on an operating table 100 with the surface of operating table 100 serving as a reference plane 103. To this end, various controls 94 are installed on computer 93, particularly image controller 110 and robot controller 120, and imaging data 110 is communicated to image controller 110 to provide an image guidance of robot manipulator 130 and intervention robot 140.

Generally, prior to patient 101 resting on operating table 100, robot manipulator 130 is affixed to operating table 100 with intervention robot 140 being spaced from operating table 100 to enable patient 101 to rest thereon.

Additionally, the robotic apparatus is registered to a interventional X-ray imager 80 as will be further explained below, and a communication 96, wired or wireless, is established between workstation 90 and robots 130 and 140.

Generally, upon patient 101 resting on operating table 100 with insertion point 102 of patient 101 being within the kinematic space of robot manipulator 130, intervention robot 140 is positioned at a starting coordinate position within the kinematic space of robot manipulator 130. Additionally, an intra-operative image of patient 101 is registered to the robotic apparatus.

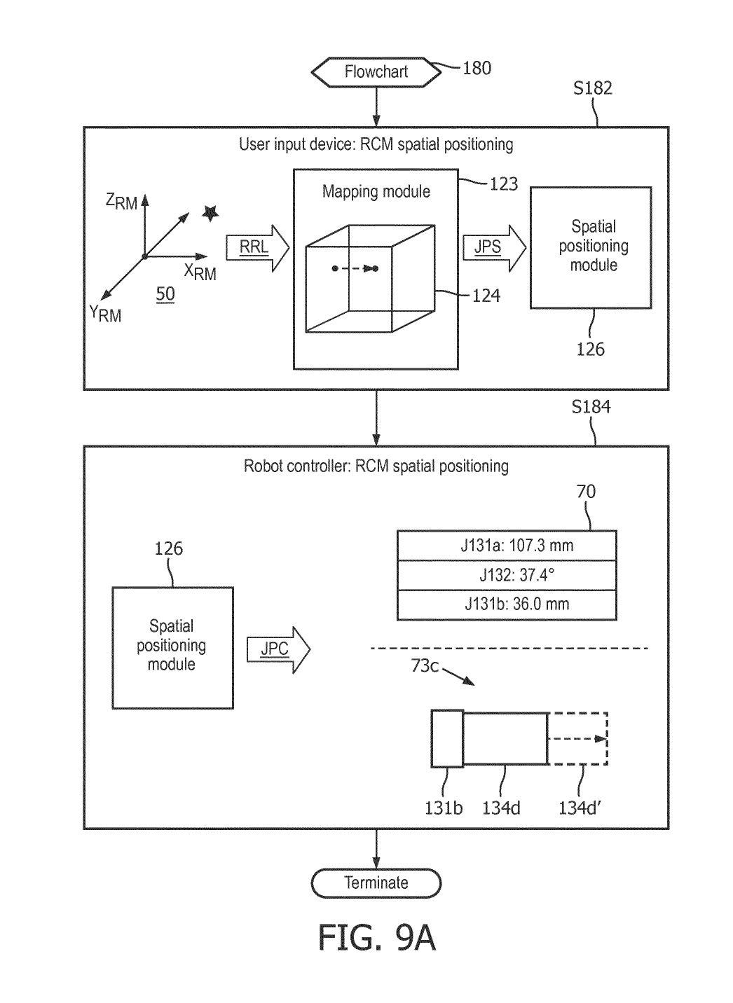

FIG. 8 illustrates a flowchart 170 representative of a robotic control method implemented by image controller 110 and robot controller 120.

Referring to FIG. 8, a stage S172 of flowchart 170 encompasses image controller 110 controlling, within the cone-beam CT image of patient 101 as generated by interventional X-ray imager 80 and displayed on monitor 91, an operator of workstation 90 delineating a planned insertion port of patient 101 as known in the art and further delineating a planned insertion angle of intervention tool 160 as known in the art.

A stage S174 of flowchart 170 encompasses image controller 110 or robot controller 120 controlling a registration of the robot apparatus to cone-beam CT image.

For embodiments of stage S174 involving a cone-beam CT image acquisition subsequent to a mounting of the robot apparatus to table 100, an automatic registration as known in the art may be executed based on a detection of an illustration of the mounted robot apparatus within the cone-beam CT image.

For embodiments of stage S174 involving a cone-beam CT image acquisition prior to a mounting of the robot apparatus to table 100 to avoid robot/imager collisions and/or image artefacts, then the cone-bean CT image may be utilized to indicate a position of the robot apparatus in a coordinate frame of fluoroscopic imager 80 whereby the indicated position is correlated to a known position of the robot apparatus via encoders or other position indicators, and the correlated position is implicitly registered through C-arm geometry to the cone-beam CT image.

Alternatively, for embodiments of stage S714 further involving camera 86, then video images generated by camera 86 may be utilized to register the robot apparatus to the cone-beam CT image by utilizing two (2) or more camera images of the robot and triangulating a position of an image based marker (e.g., an image pattern) of the robot apparatus with a known relationship to the robot coordinate frame, or if the robot apparatus is not equipped with an image based, marker, by utilizing video images of camera 86 to indicate a position of the robot apparatus in a coordinate frame of fluoroscopic imager 80 whereby the indicated position is correlated to a known position of the robot apparatus via encoders or other position indicators, and the correlated position is implicitly registered through C-arm geometry to the cone-beam CT image.

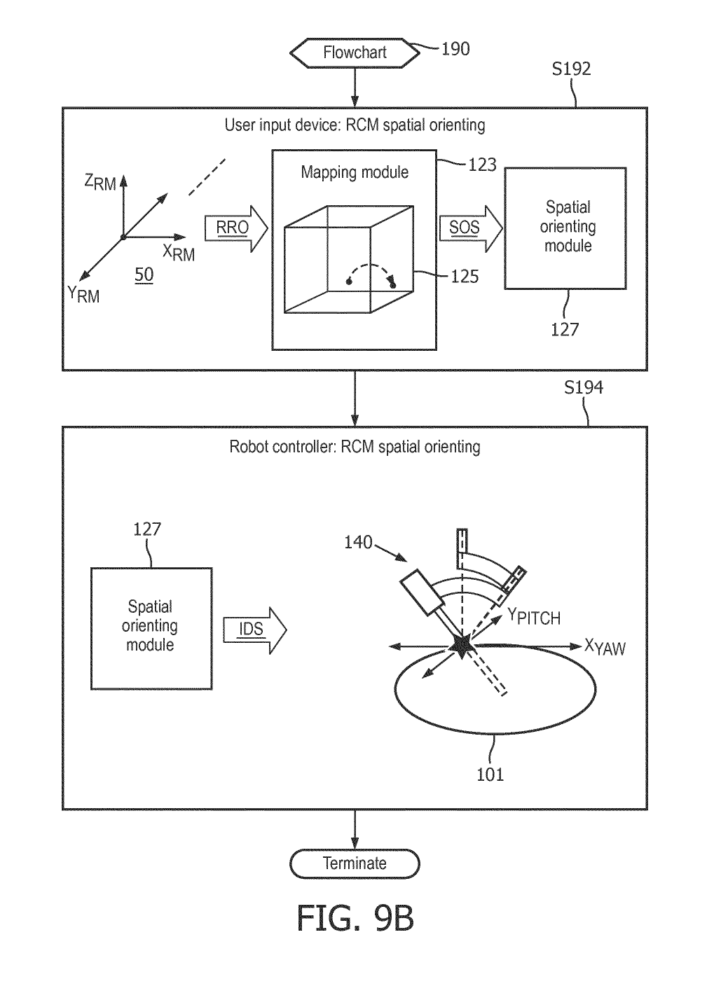

A stage S176 of flowchart 170 encompasses a manual actuation by the operator of workstation 90 of robot manipulator 130 of a translation motion and/or a rotational motion of robot manipulator 130 as necessary in a direction of insertion point 102 of a patient 101 to spatially position intervention robot 140 within the kinematic space of robot manipulator 130. To this end, as shown in FIG. 9A, robot controller 120 is operated to execute a flowchart 180 representative of a robot manipulator control method of the inventions of the present disclosure.

Referring to FIG. 9A, a stage S182 of flowchart 180 encompasses mapping module 123 of robot controller 120 processing a registered robot location RRL indicative of the spatial positioning of the remote-center-of-motion RCM at insertion point 102 within cone-beam CT image as registered to the robot apparatus. The processing involves a computation of joint position settings JPS in accordance with a mapping 124 by mapping module 123 of the registered robot location RRL within kinematic space 150 of robot manipulator 130, and a communication of joint position settings JPS to spatial positioning module 126.

A stage S184 of flowchart 180 encompasses spatial positioning module 126 executing joint position commands JPC to thereby facilitate a manual actuation of a translational and/or a rotational motion as needed of robot manipulator 150 as affixed to reference plane 103. Examples of joint position commands JPC include, but are not limited to, textual display 70 and/or graphical images 73 as previously described herein.

Referring to back to FIG. 8, upon completion of stage S176, a stage S178 of flowchart 170 encompasses a signal actuation by robot manipulator 130 of a pitch motion and/or a yaw motion of intervention robot 140 as necessary about insertion point 102 of a patient 101 to spatially orient end-effector 145 (FIG. 6) within the kinematic space of intervention robot 140 (FIG. 6). To this end, as shown in FIG. 9B, robot controller 120 is operated to execute a flowchart 190 representative of an intervention robot control method of the inventions of the present disclosure.

Referring to FIG. 9B, a stage S192 of flowchart 190 encompasses mapping module 123 of robot controller 120 processing a registered robot orientation RRO indicative of the spatial orienting of the remote-center-of-motion RCM about insertion point 102 within cone-beam CT image as registered to the robot apparatus. The processing involves a generation of spatial orienting signal SOS in accordance with a mapping 125 by mapping module 123 of the registered robot orientation RRO within kinematic space 150 of intervention robot 140, and a communication of spatial orienting signal SOS to spatial orientating module 127.

A stage S194 of flowchart 190 encompasses spatial orientating module 127 transmitting intervention drive signal IDS to the actuator(s) of intervention robot 140 to thereby pitch and/to yaw intervention robot 140 as needed.

Flowchart 170 is terminated upon completion of stage S194.

Referring to FIGS. 1-9, those having ordinary skill in the art will appreciate numerous benefits of the present disclosure including, but not limited to, a decoupled kinematics providing independent control of an insertion point and an insertion angle offering many advantages including accuracy and intuitiveness of control of a robotic apparatus, and image guidance allowing operator selection of the insertion point and the insertion angle from diagnostic images further offering accuracy and intuitiveness of control of a robotic apparatus.

Furthermore, as one having ordinary skill in the art will appreciate in view of the teachings provided herein, features, elements, components, etc. described in the present disclosure/specification and/or depicted in the Figures may be implemented in various combinations of electronic components/circuitry, hardware, executable software and executable firmware and provide functions which may be combined in a single element or multiple elements. For example, the functions of the various features, elements, components, etc. shown/illustrated/depicted in the Figures can be provided through the use of dedicated hardware as well as hardware capable of executing software in association with appropriate software. When provided by a processor, the functions can be provided by a single dedicated processor, by a single shared processor, or by a plurality of individual processors, some of which can be shared and/or multiplexed. Moreover, explicit use of the term “processor” should not be construed to refer exclusively to hardware capable of executing software, and can implicitly include, without limitation, digital signal processor (“DSP”) hardware, memory (e.g., read only memory (“ROM”) for storing software, random access memory (“RAM”), non-volatile storage, etc.) and virtually any means and/or machine (including hardware, software, firmware, circuitry, combinations thereof, etc.) which is capable of (and/or configurable) to perform and/or control a process.

Moreover, all statements herein reciting principles, aspects, and embodiments of the invention, as well as specific examples thereof, are intended to encompass both structural and functional equivalents thereof. Additionally, it is intended that such equivalents include both currently known equivalents as well as equivalents developed in the future (e.g., any elements developed that can perform the same or substantially similar function, regardless of structure). Thus, for example, it will be appreciated by one having ordinary skill in the art in view of the teachings provided herein that any block diagrams presented herein can represent conceptual views of illustrative system components and/or circuitry embodying the principles of the invention. Similarly, one having ordinary skill in the art should appreciate in view of the teachings provided herein that any flow charts, flow diagrams and the like can represent various processes which can be substantially represented in computer readable storage media and so executed by a computer, processor or other device with processing capabilities, whether or not such computer or processor is explicitly shown.

Furthermore, exemplary embodiments of the present disclosure can take the form of a computer program product or application module accessible from a computer-usable and/or computer-readable storage medium providing program code and/or instructions for use by or in connection with, e.g., a computer or any instruction execution system. In accordance with the present disclosure, a computer-usable or computer readable storage medium can be any apparatus that can, e.g., include, store, communicate, propagate or transport the program for use by or in connection with the instruction execution system, apparatus or device. Such exemplary medium can be, e.g., an electronic, magnetic, optical, electromagnetic, infrared or semiconductor system (or apparatus or device) or a propagation medium. Examples of a computer-readable medium include, e.g., a semiconductor or solid state memory, magnetic tape, a removable computer diskette, a random access memory (RAM), a read-only memory (ROM), flash (drive), a rigid magnetic disk and an optical disk. Current examples of optical disks include compact disk read only memory (CD-ROM), compact disk read/write (CD-R/W) and DVD. Further, it should be understood that any new computer-readable medium which may hereafter be developed should also be considered as computer-readable medium as may be used or referred to in accordance with exemplary embodiments of the present disclosure and disclosure.

Having described preferred and exemplary embodiments of novel and inventive image guidance of robotic systems, (which embodiments are intended to be illustrative and not limiting), it is noted that modifications and variations can be made by persons having ordinary skill in the art in light of the teachings provided herein, including the FIGS. 1-9. It is therefore to be understood that changes can be made in/to the preferred and exemplary embodiments of the present disclosure which are within the scope of the embodiments disclosed herein.

Moreover, it is contemplated that corresponding and/or related systems incorporating and/or implementing the device or such as may be used/implemented in a device in accordance with the present disclosure are also contemplated and considered to be within the scope of the present disclosure. Further, corresponding and/or related method for manufacturing and/or using a device and/or system in accordance with the present disclosure are also contemplated and considered to be within the scope of the present disclosure.

1. A robotic system for executing an interventional procedure, the robotic system comprising:
a robotic apparatus including
a robot manipulator, and
an intervention robot mounted to the robot manipulator, wherein the intervention robot includes an end-effector and a structural configuration of the intervention robot defines a remote-center-of-motion; and
a robot controller,
wherein the robot controller controls a manual actuation of at least a translational motion and a rotational motion of the robot manipulator directed to a spatial positioning of the intervention robot within a kinematic space of the robot manipulator derived from a delineation of a spatial positioning of the remote-center-of-motion within an image space, and
wherein the robot controller controls a signal actuation of at least one of a pitch motion and a yaw motion of the intervention robot directed to a spatial orienting of the end-effector within a kinematic space of the intervention robot derived from a delineation of a spatial orienting of the remote-center-of-motion within the image space.

2. The robotic system of claim 1, wherein the robot controller controls the delineations of the spatial positioning and the spatial orienting of the remote-center-of-motion within the image space.

3. The robotic system of claim 1, further comprising:
an image controller,
wherein the image controller controls a communication to the robot controller of the delineations of the spatial positioning and the spatial orienting of the remote-center-of-motion within the image space.

4. The robotic system of claim 3, wherein the positioning communication from the image controller to the robot controller includes at least one of
image data illustrative of the delineation of the spatial positioning and the spatial orienting of the remote-center-of-motion within the image space; and
coordinate data informative of the delineation of the spatial positioning and the spatial orienting of the remote-center-of-motion within the image space.

5. The robotic system of claim 1, wherein the control by the robot controller of the manual actuation of the at least the translational motion and the rotational motion of the robot manipulator includes:
at least one of a textual display, an audible broadcast and a graphical image indicative of the spatial positioning of the intervention robot within a kinematic space of the robot manipulator.

6. The robotic system of claim 1, wherein the robot manipulator includes at least one of a linear encoder and a rotary encoder for monitoring a spatial position of the intervention robot within the kinematic space of the robot manipulator.

7. The robotic system of claim 1, wherein the robot manipulator includes at least one of a linear measurer and an angular measurer for monitoring the spatial positioning of the intervention robot within the kinematic space of the robot manipulator.

8. The robotic system of claim 1, wherein the robot manipulator includes at least one prismatic joint manually translatable for the manual actuation of the translational motion of the robot manipulator directed to the spatial positioning of the intervention robot within the kinematic space of the robot manipulator.

9. The robotic system of claim 1, wherein the robot manipulator includes at least one revolute joint manually rotatable for the manual actuation of the rotational motion of the robot manipulator directed to the spatial positioning of the intervention robot within the kinematic space of the robot manipulator.

10. The robotic system of claim 1, wherein the robot manipulator includes:
at least two prismatic joints manually translatable for the manual actuation of the translational motion of the robot manipulator directed to the spatial positioning of the intervention robot within the kinematic space of the robot manipulator; and
at least one revolute joint manually rotatable for the manual actuation of the rotational motion of the robot manipulator directed to the spatial positioning of the intervention robot within the kinematic space of the robot manipulator.

11. The robotic system of claim 1,
wherein the intervention robot includes at least one revolute joint; and
wherein the robot controller is operably connected to the at least one revolute joint to drive the at least one of the pitch motion and the yaw motion of the intervention robot.

12. The robotic system of claim 1,
wherein the intervention robot further includes
at least two revolute joints
wherein the remote-center-of-motion is a point coinciding with an intersection of an axis of each of the at least two revolute joints and an axis of the end-effector; and
wherein the robot controller is operably connected to the at least two revolute joints to drive the at least one of the pitch motion and the yaw motion of the intervention robot.

13. A controller network for controlling a robotic apparatus including an intervention robot mounted unto a robot manipulator and a structural configuration of the intervention robot defining a remote-center-of-motion, the controller network comprising:
a robot controller,
wherein the robot controller controls a manual actuation of at least a translational motion and a rotational motion of the robot manipulator directed to a spatial positioning of the intervention robot within a kinematic space of the robot manipulator derived from a delineation of a spatial positioning of the remote-center-of-motion within an image space, and
wherein the robot controller controls a signal actuation of at least one of a pitch motion and a yaw motion of the intervention robot directed to a spatial orienting of the end-effector within a kinematic space of the intervention robot derived from a delineation of a spatial orienting of the remote-center-of-motion within the image space; and
an image controller,
wherein the image controller controls a positioning communication to the robot controller of the delineations of the spatial positioning and the spatial orienting of the remote-center-of-motion within the image space.

14. The controller network of claim 13, wherein the positioning communication from the image controller to the robot controller includes at least one of
image data illustrative of the delineation of the spatial positioning and the spatial orienting of the remote-center-of-motion within the image space; and
coordinate data informative of the delineation of the spatial positioning and the spatial orienting of the remote-center-of-motion within the image space.

15. The controller network of claim 13, wherein the robot controller and the image controller are installed within a same workstation.

16. A method for controlling a robotic apparatus including an intervention robot mounted unto a robot manipulator and a structural configuration of the intervention robot defining a remote-center-of-motion, the method comprising:
a robot controller controlling a manual actuation of at least a translational motion and a rotational motion of the robot manipulator directed to a spatial positioning of the intervention robot within a kinematic space of the robot manipulator derived from a delineation of a spatial positioning of the remote-center-of-motion within an image space; and
the robot controller controlling a signal actuation of at least one of a pitch motion and a yaw motion of the intervention robot directed to a spatial orienting of the end-effector within a kinematic space of the intervention robot derived from a delineation of a spatial orienting of the remote-center-of-motion within the image space.

17. The method of claim 16, further comprising:
the robot controller controlling the delineation of the spatial positioning and the spatial orienting of the remote-center-of-motion within the image space.

18. The method of claim 16, further comprising:
an image controller controlling a positioning communication to the robot controller of the delineation of the spatial positioning and the spatial orienting of the remote-center-of-motion within the image space.

19. The method of claim 18, wherein the positioning communication from the image controller to the robot controller includes at least one of
image data illustrative of the delineation of the spatial positioning and the spatial orienting of the remote-center-of-motion within the image space; and
coordinate data informative of the delineation of the spatial positioning and the spatial orienting of the remote-center-of-motion within the image space.

20. The method of claim 16, wherein the control by the robot controller of the manual actuation of the at least the translational motion and the rotational motion of the robot manipulator includes:
at least one of a textual display, an audible broadcast and a graphical image indicative of the spatial positioning of the intervention robot within a kinematic space of the robot manipulator.
